(19) United States
(12) Patent Application Publication
(10) Pub. No.: US 2014/0057829 A1
(43) Pub. Date: Feb. 27, 2014

(54) Citrullinated Histone H3 (Cit H3) in Septic Shock

(71) Applicants: Hasan B. Alam, Ann Arbor, MA (US); Yongqing Li, West Roxbury, MA (US)

(72) Inventors: Hasan B. Alam, Ann Arbor, MA (US); Yongqing Li, West Roxbury, MA (US)

(73) Assignee: THE GENERAL HOSPITAL CORPORATION, Boston, MA (US)

(21) Appl. No.: 13/981,032

(22) PCT Filed: Feb. 1, 2012
(86) PCT No.: PCT/US12/23435

§ 371 Date: Nov. 6, 2013

Related U.S. Application Data
(60) Provisional application No. 61/438,377, filed on Feb. 1, 2011.

(51) Int. Cl. G01N 33/68

(52) U.S. Cl. CPC G01N 33/6893
USPC 514/1.4; 435/7.92; 514/192; 514/200; 514/23; 514/312; 514/376

(57) ABSTRACT

Methods of diagnosing sepsis, severe sepsis, or septic shock and predicting prognosis in subjects with septic shock, based on levels of citrullinated histone H3 (Cit H3) in the subject, e.g., in a sample comprising serum (e.g., whole blood, serum, or plasma), cerebrospinal fluid, urine, saliva, or peritoneal fluid from the subject.

### CLAIM OF PRIORITY

This application claims the benefit of U.S. Provisional Patent Application Ser. No. 61/438,377, filed on Feb. 1, 2011, the entire contents of which are hereby incorporated by reference.

### FEDERALLY SPONSORED RESEARCH OR DEVELOPMENT

This invention was made with Government support under Grant No. RO1 GM084127 awarded by the National Institutes of Health. The Government has certain rights in the invention.

### TECHNICAL FIELD

This invention relates to methods of diagnosing septic shock and predicting prognosis in subjects with septic shock, based on levels of citrullinated histone H3 (Cit H3) in the subject, e.g., in serum (e.g., whole blood, serum, or plasma), cerebrospinal fluid, urine, saliva, or peritoneal fluid from the subject.

### BACKGROUND

Sepsis is a systemic inflammatory disorder and its progression to septic shock is a serious clinical problem with very high mortality. Early warning signs are frequently nonspecific and inconspicuous. Preceding symptoms can easily be mistaken as due to non-infected etiologies. The effectiveness of blood culture is usually considered a “gold standard” for diagnosing septicemia. However, a positive culture may take more than 48 hours of incubation and false negative results are very common due to the low density of blood bacteria at the early stage of infection. Alternative markers with higher sensitivity, specificity, and predictive value could help in the early detection and monitoring of sepsis progression, as well as the response to treatment. Therefore, identification of prognostic markers that predict risk of developing septic shock would help in the management of subjects with sepsis.

### SUMMARY

The present invention is based, at least in part, on the discovery that Cit H3 is released into circulation during the early stages of LPS-induced shock, and that levels of Cit H3 are significantly associated with severity of LPS-induced shock. Therefore, Cit H3 can be used for early diagnosis of sepsis, severe sepsis, and septic shock, and for predicting outcome, e.g., lethality, of sepsis, severe sepsis, or septic shock.

In a first aspect, the present invention provides methods, e.g., in vitro methods, of predicting prognosis of a subject suffering from septic shock. The methods include determining a level of Cit H3 in a sample comprising serum from the subject to obtain a test value; and comparing the test value to a reference value, wherein a test value compared to the reference value indicates the subject's prognosis.

In some embodiments, the reference value represents a threshold level of Cit H3, wherein the presence of a level of Cit H3 in the subject that is above the reference value indicates that the subject has an increased risk of mortality, complications, or longer hospitalization due to septic shock, and the presence of a level of Cit H3 in the subject that is below the reference value indicates that the subject has an increased chance of survival.

In a further aspect, the present invention provides methods, e.g., in vitro methods, of predicting prognosis of a subject suffering from Systemic Inflammatory Response Syndrome (SIRS), sepsis, or severe sepsis. The methods include determining a level of Cit H3 in a sample comprising serum from the subject to obtain a test value; and comparing the test value to a reference value. A test value compared to the reference value indicates the subject's prognosis.

In some embodiments of the methods described herein, the reference value represents a threshold level of Cit H3, wherein the presence of a level of Cit H3 in the subject that is above the reference value indicates that the subject has an increased risk of progressing to severe sepsis or septic shock, and the presence of a level of Cit H3 in the subject that is below the reference value indicates that the subject does not have an increased risk of progressing to severe sepsis or septic shock.

In an additional aspect, the present invention provides methods, e.g., in vitro methods, of diagnosing sepsis, severe sepsis, or septic shock in a subject suffering from Systemic Inflammatory Response Syndrome (SIRS). The methods include determining a level of Cit H3 in a sample comprising serum from the subject to obtain a test value; and comparing the test value to a reference value. A test value compared to the reference value indicates whether the subject has sepsis, severe sepsis, or septic shock.

In some embodiments of the methods described herein, the reference value represents a threshold level of Cit H3, wherein the presence of a level of Cit H3 in the subject that is above the reference value indicates that the subject has septic shock, and the presence of a level of Cit H3 in the subject that is below the reference value indicates that the subject does not have septic shock. In some embodiments of the methods described herein, the methods further include selecting a subject who has a level of Cit H3 in the subject that is above the reference value, and administering one or more treatments for septic shock to the subject.

In some embodiments, the treatment comprises administering a therapeutically effective amount of an antibiotic.

In another aspect, the present invention provides methods, e.g., in vitro methods, for evaluating the efficacy of a treatment for septic shock (SS) in a subject. The methods include determining a level of Cit H3 in a first sample comprising serum from the subject to obtain a first value; administering a treatment for septic shock to the subject; determining a level of Cit H3 in a subsequent sample comprising serum obtained from the subject at a later time, to obtain a treatment value; and comparing the first value to the treatment value. A treatment value that is below the first value indicates that the treatment is effective. In some embodiments, the first and second samples comprise plasma or whole blood.

In some embodiments of the methods described herein, the treatment includes administration of an effective amount of an antibiotic.

In another aspect, the present invention provides methods, e.g., in vitro methods, for determining an effect of a treatment for septic shock (SS) on prognosis in a subject. The methods include determining a level of Cit H3 in a first sample comprising serum from the subject to obtain a first value; administering a treatment for septic shock to the subject; determining a level of Cit H3 in a subsequent sample comprising serum obtained from the subject at a later time to obtain a treatment value; and comparing the first value to the treatment value. A decrease in the level of Cit H3 from the first value to the treatment value indicates that the treatment has improved the subject's prognosis, and an increase or no change in the level of Cit H3 indicates that the treatment has not affected or has worsened the subject's prognosis. In some embodiments of the methods described herein, the first and second samples comprise plasma or whole blood.

In some embodiments of the methods described herein, the treatment includes administration of an effective amount of an antibiotic.

In a further aspect, the present invention provides methods, e.g., in vitro methods, for determining severity of septic shock in a subject, the method comprising: determining a level of Cit H3 in a sample comprising serum from the subject to obtain a test value; and comparing the test value to a reference value. A test value compared to the reference value indicates the severity of septic shock in the subject.

In some embodiments of the methods described herein, the reference value represents a threshold level of Cit H3, wherein the presence of a level of Cit H3 in the subject that is above the reference value indicates that the subject has severe septic shock, and the presence of a level of Cit H3 in the subject that is below the reference value indicates that the subject does not have severe septic shock.

In some embodiments of the methods described herein, the subject is a mammal, e.g., a human, or a non-human veterinary subject or experimental animal.

In some embodiments of the methods described herein, levels of Cit H3 are measured in a sample from the subject, e.g., a sample comprising serum (e.g., whole blood, serum, or plasma), cerebrospinal fluid, urine, saliva, or peritoneal fluid from the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B show that serum levels of histone H3 in the early stage of septic shock of mice treated with or without SAHA.

1A, representative Western blots of mouse serum blotted with anti-histone H3 antibody (top panel) or control IgG (bottom panel) from animal groups of sham, LPS and SAHA+LPS (n=3 each). Blood was collected at 3 h after LPS injection. Sham (no LPS, and no SAHA treatment) animals serve as a control. 1B, a bar graph showing results when the specific histone H3 bands were quantified by densitometry and expressed as mean values ±SD (n=3). The symbol * indicates significant difference from LPS group (p<0.001).

FIGS. 2A-C show that SAHA decreases expression and secretion of citrullinated histone H3 (Cit H3) protein in LPS-stimulated HL-60 granulocytes.

2A, images of four representative Western blots of HL-60 cell lysates with antibodies against histone H3 (H3), acetyl histone H3 (Ac H3) and citrullinated histone H3 (Cit H3) from groups of sham, LPS and SAHA+LPS (n=3/group). Actin serves as an internal control.

2B, a bar graph showing the results when protein bands of Cit H3, H3 and Ac H3 were scanned, quantified by densitometry and expressed as mean values ±SD (n=3/group). The symbol * indicates that a value significantly differs from sham and SAHA+LPS groups (p<0.05). The symbol # indicates that a value significantly differs from sham and LPS groups (p<0.001).

2C, a set of four images of western blots showing the results when media from cultured HL-60 cells was separated on SDS-PAGE and analyzed by Western blotting with antibodies against H3, Cit H3, and IgG (control).

FIGS. 3A-D show that serum levels of citrullinated histone H3 (Cit H3) and histone H3 (H3) in mice underwent different insults.

3A, images showing results of representative Western blotting of mouse serum with antibodies against H3, Ac H3 and Cit H3 from animal groups of sham, hemorrhagic shock (HS), LPS small dose (SD) and LPS large dose (LD) (n=5/group). Immunoglobulin (Ig) serves as an internal control.

3B, a bar graph showing the results when specific Cit H3 bands were scanned, quantified by densitometry and expressed as mean values ±SD (n=5/group). The symbol * indicates that a value significantly differs from sham and LPS (SD) groups (p<0.001).

3C, a bar graph showing the results when specific H3 bands were scanned, quantified by densitometry and expressed as mean values ±SD (n=5/group). The symbol * indicates that a value significantly differs from Sham, Hem (hemorrhagic shock) and LPS (SD) groups (p<0.009). The symbol # indicates that a value significantly differs from Sham (p<0.038), and Hem groups (p<0.016).

3D, Kaplan-Meier curves comparing survival between the LPS (LD) and LPS (SD) groups. Mice were intraperitoneally injected with LPS (SD) (10 mg/kg) or LPS (LD) (35 mg/kg) (n=10 per group). Survival rates were recorded over 168 hours (7 days) after LPS insult and statistical analysis was performed. The symbol “arrow” indicates blood-drawing time (3 h after LPS injection). The symbol * indicates that a value significantly differs from the LPS (LD) group (p<0.01).

FIG. 4 is a bar graph showing no significant difference of serum TNF-α between LPS (SD) and LPS (LD) groups. Blood was collected from sham, LPS (SD), and LPS (LD) groups at 3 h after LPS injection (n=5/group) and serums were prepared from these blood samples. The level of TNF-α protein was analyzed by ELISA. All analyses were performed in triplicate. No significant difference was found between LPS (SD) and LPS (LD) groups (p=0.77).

FIG. 5 is a schematic illustration of possible sources of serum citrullinated histone H3 (Cit H3) and histone H3 (H3). Without wishing to be bound by theory, serum H3 and Cit H3 could come from dying cells and Neutrophil Extracellular Traps (NETs). The circulating histone proteins may result from formation of NETs during LPS-induced sepsis. At the beginning of sepsis, LPS stimulates histone citrullination catalyzed by peptidyl arginine deiminase 4 (PAD4) in neutrophils. These neutrophils enter a program where the nuclear and granular membranes dissolve, and the nuclear contents decondense into the cytoplasm. Then the plasma membrane ruptures and chromatin is released into the extracellular space.

FIG. 6 is a set of four Western Blots showing the results of detection of Cit H3 protein in human blood from healthy volunteers (Control (−)), hemorrhagic trauma patients (Trauma), and septic patients (Patient 1.2 and Patient 1.3) (top panels); IgG was also detected as a loading control (bottom panels). Plasma samples of mouse septic shock served as a positive (+) control.

### DETAILED DESCRIPTION

As described herein, improved survival in LPS injected animals that are treated with SAHA is associated with a reduction in H3 protein levels. LPS stimulates histone H3 deimination/citrullination in HL-60 granulocytes and in an in-vivo mouse model of septic shock. Moreover, H3 deimination induced by LPS can enhance HL-60 neutrophil secretion of histone proteins (H3 and Cit H3) into the extracellular space, suggesting that Cit H3 could at least in part initiate the formation of NETs and lead to an increase in histone proteins. Finally, levels of Cit H3 correlate with the severity of LPS-induced sepsis, which indicates that early measurement of circulating Cit H3 protein can be helpful in predicting survival in lethal septic shock.

Histones

Histones are nuclear proteins that are the major protein component of chromatin, providing a structural core around which nuclear DNA is packaged. Posttranslational modification of a histone protein, such as deimination (arginine to citrulline conversion, also referred to as citrullination) and acetylation, can change its structure and function. Deimination/citrullination of histones by PAD4, which has been shown to target multiple arginine sites in histones H3 (Arg-2, Arg-8, Arg-17, and Arg-26, e.g., Arg-8 and Arg-17; see, e.g., Bauer et al., (2002) EMBO Rep, 3:39-44, and Wang et al. Science 306:279-283, (2004)), can be induced by lipopolysaccharide (LPS). PAD4 is an enzyme previously known to convert protein arginine (Arg) to citrulline (Cit), a nonconventional amino acid in proteins (Vossenaar et al., Bioessays. 2003; 25: 1106-1118). This enzyme was first identified in human HL-60 leukemia cells upon differentiation along the granulocyte lineage (Nakashima et al., J Biol Chem. 1999; 274(39): 27786-27792) and is highly expressed in peripheral blood neutrophils (Nakashima et al., J Biol Chem. 2002; 277(51): 49562-49568). It is known that the LPS-induced histone deimination/citrullination is an early response to inflammatory stimuli in neutrophils (Neeli et al., J Immunol. 2008; 180(3): 1895-1902). Moreover, citrullinated histone H3 (Cit H3) has been identified as a component of neutrophil extracellular traps (NETs) that are produced by degranulating neutrophils. This Cit H3 is released in the extracellular space as part of the neutrophiil response to infection (Neeli et al., J Innate Immun. 2009; 1(3): 194-201).

The accumulated data point towards a link between citrullinated proteins and pathogenesis of diseases such as rheumatoid arthritis, multiple sclerosis and psoriasis. Lundberg et al showed that appearance and amounts of citrullinated proteins in arthritic joints of experimental animals are correlated with severity of inflammation (Lundberg et al., Arthritis Res Ther. 2005; 7(3): R458-467). However, these proteins reflect their relationship with autoimmune diseases. It remains unknown whether a citrullinated protein in neutrophils could serve as a biomarker to predict the severity of sepsis.

Recently, Xu et at (Nat Med. 2009; 15(11): 1318-1321) reported their findings in Nature Medicine and stated that “extracellular histones, mainly H3 and H4, seem to be both biomarkers of disease progression and therapeutic targets in sepsis . . . ”. In the present study, we measured histone H3 as a positive control, and additionally evaluated circulating levels of Cit H3 after lipopolysaccharide (LPS) injection in rodent model. We have already shown that treatment with suberoylanilide hydroxamic acid (SAHA), a histone deacetylase inhibitor, in rodent models of LPS-induced septic shock improves survival (Li et al., Shock. 2009; 32(5): 517-523; Li et al., Surgery. 2010; 148(2): 246-254). First, serum from these animals was screened for circulating levels of histone proteins. Second, a possible source of the histone proteins was assessed in serum using a model of HL-60 granulocytes in vitro. Finally, whether the serum Cit H3 protein associates with severity of sepsis (and outcome) was determined using lethal and sub-lethal doses of LPS in vivo.

Diagnosis and Prognosis

The methods described herein can be used to diagnose the presence of, and predict the outcome (prognosis) of sepsis, severe sepsis, and septic shock. Septic shock is the presence of infection associated with a systemic inflammatory response that results in physiologic alterations at the capillary endothelial level, manifesting as a drop in blood pressure. Septic shock can be caused by any type of bacteria, as well as some fungi and viruses. Most common in the elderly and very young children and infants, septic shock also occurs in people who suffer other illnesses, including diabetes, immune system disorders such as AIDS, diseases of the genitourinary, biliary, or intestinal tracts, leukemia, or lymphoma, or who have indwelling long-term catheters, recent surgeries, or use of steroids or antibiotics.

Outward symptoms of septic shock include, e.g., reduced urine output (e.g., oliguria or anuria), cool, pale extremities; high or very low temperature, chills; lightheadedness; low blood pressure, especially when standing; low or absent urine output; palpitations; rapid heart rate; restlessness, agitation, lethargy, or confusion; shortness of breath; and skin rash or discoloration. However, these typical signs and symptoms are seen in advanced stages of SS. Early diagnosis is often difficult, and use of sensitive biomarkers that can identify shock before it becomes clinically apparent can result in early administration of life saving therapies. Blood tests may be done to check for infection, low blood oxygen level, abnormal acid-base balance, or poor organ function or organ failure; a chest x-ray may be used to detect pneumonia or pulmonary edema; and/or a urine sample may be taken to detect infection. Blood cultures, may not become positive for several days after the blood has been taken, or for several days after the shock has developed. See, e.g., Vincent, “Septic Shock.” In: Fink et al., eds. Textbook of Critical Care. 5th ed. Philadelphia, Pa.: Saunders Elsevier; 2005: chap 147; Jones and Kline, “Shock.” In: Marx, ed. Rosen's Emergency Medicine: Concepts and Clinical Practice. 6th ed. Philadelphia, Pa.: Mosby Elsevier; 2006: chap 4; Munford. “Severe sepsis and septic shock.” In: Fauci and Harrison, eds. Harrison's Principles of Internal Medicine. 17th ed. New York, N.Y.: McGraw Hill; 2008:chap 265.

Given that in the absence of an obvious infection, an absolute diagnosis of sepsis, severe sepsis, or septic shock (i.e., a diagnosis confirmed by the presence of pathogens in a blood or urine culture) can thus be delayed by several days, and that other conditions—such as traumatic shock and Systemic Inflammatory Response Syndrome (SIRS) in the absence of infection—present with the same clinical symptoms, the present methods allow for the rapid and early diagnosis of sepsis, severe sepsis, septic shock in these subjects who can then be treated promptly with antibiotic or antimicrobial therapy. SIRS is diagnosed in the presence of two or more of the following conditions:

Temperature >38° C. or <36° C.

Heart rate >90 beats/min

Respiratory rate >20 breaths/min or PaCO2<32 mm Hg

WBC count >12,000/mm³, <4000/mm³, or >10% immature (band) forms.

Typically, sepsis is defined by the same criteria as SIRS, plus the presence of a documented infection. Severe sepsis is defined by the criteria of sepsis plus organ dysfunction, hypoperfusion, or hypotension. Hypoperfusion and perfusion abnormalities can include, but are not limited to, lactic acidosis, oliguria, or an acute alteration in mental status. Septic shock is severe sepsis plus hypotension despite adequate fluid resuscitation and perfusion abnormalities that can include, but are not limited to, lactic acidosis, oliguria, or an acute alteration in mental status. Subjects who have been treated with inotropics or vasopressors and are essentially normotensive, but who have hypoperfusion abnormalities or organ dysfunction, could be diagnosed with septic shock.

As demonstrated herein, the presence of sepsis, severe sepsis, or septic shock causes an acute rise in Cit H3 protein levels. Further, SAHA treatment attenuates these alterations and stabilizes Cit H3 levels. The results demonstrate that levels of Cit H3 are a biomarker for sepsis, severe sepsis, or septic shock diagnosis, and for successful drug treatment of sepsis, severe sepsis, or septic shock. In addition, levels of Cit H3 are correlated with severity of septic shock, and thus can be used to determine severity of disease and prognosis.

Individuals considered at risk for septic shock may benefit particularly from the methods described herein, primarily because once an elevated level of Cit H3 is detected, e.g., in a subject who is at risk for septic shock, early treatment can begin before there is any clinical evidence of septic shock. Individuals “at risk” include, e.g., individuals suffering from any condition described above, e.g., sepsis or severe sepsis, or having another factor that may put a patient at risk for severe infection, e.g., a chronic or hereditary disorder (e.g., SCID) or because of immune-suppressive medical treatments (e.g., chemotherapy or steroids). For example, a person suffering from an infection can be diagnosed according to the methods described herein and treated before full-blown septic shock occurs.

Skilled practitioners will appreciate that a patient can be identified as at risk for septic shock by any method known in the art, e.g., by a physician or other medical personnel.

In some embodiments, the methods of diagnosis described herein are performed in conjunction with a standard septic shock workup, e.g., including laboratory and other tests (e.g., as described above, plus complete blood count (CBC); prothrombin time and/or activated partial thromboplastin time; urine output rate; arterial blood gases (ABG) (levels reflect acid-base and perfusion status); and lactate and base deficit (used in some centers to indicate the degree of metabolic debt; clearance of these markers over time can reflect the adequacy of resuscitation). Imaging studies, e.g., (standard radiography, computed tomography, ultrasonography, and directed angiography), an ECG, or tissue oximetry can also be used.

Methods for diagnosing sepsis, severe sepsis, or septic shock include determining a level of Cit H3 in the of the subject to obtain a Cit H3 value, and comparing the value to an appropriate reference value, e.g., a value that represents a threshold level, above which the subject can be diagnosed with sepsis, severe sepsis, or septic shock. The reference can also be a range of values, e.g., that indicate severity of sepsis, severe sepsis, or septic shock in the subject. A suitable reference value can be determined by methods known in the art.

Therefore, included herein are methods for diagnosing sepsis, severe sepsis, or septic shock in a subject. The methods include obtaining a sample from a subject, and evaluating the presence and/or level of Cit H3 in the sample, and comparing the presence and/or level with one or more references, e.g., a control reference that represents a normal level of Cit H3, e.g., a level in an unaffected subject, and/or a disease reference that represents a level of Cit H3 associated with sepsis, severe sepsis, or septic shock, e.g., a level in a subject having sepsis, severe sepsis, or septic shock.

In some embodiments, the presence and/or level of Cit H3 is comparable to the presence and/or level of the protein(s) in the disease reference, and the subject has one or more symptoms associated with sepsis, severe sepsis, or septic shock, then the subject has sepsis, severe sepsis, or septic shock. For example, in a subject who has SIRS and elevated Cit H3 levels (i.e., levels above a disease reference level), sepsis can be diagnosed. In a subject who has elevated Cit H3 levels and SIRS, plus organ dysfunction, hypoperfusion, or hypotension, severe sepsis can be diagnosed. In a subject who has severe sepsis plus hypotension despite adequate fluid resuscitation and perfusion abnormalities that can include, but are not limited to, lactic acidosis, oliguria, or an acute alteration in mental status, the presence of elevated Cit H3 levels indicates a diagnosis of septic shock.

In some embodiments, the subject has no overt signs or symptoms of septic shock, but the presence and/or level of one or more of the proteins evaluated is comparable to the presence and/or level of the protein(s) in the disease reference, then the subject has an increased risk of developing septic shock. In some embodiments, once it has been determined that a person has septic shock, or has an increased risk of developing septic shock, then a treatment, e.g., as known in the art or as described herein, can be administered. The efficacy of the treatment can be monitored using the methods described herein.

As noted above, levels of Cit H3 are correlated with severity of disease; thus, the methods can include comparing the level of Cit H3 in a sample from a subject to a reference level or a range of reference levels that represent (are correlated with) differing degrees of severity, thereby determining the severity of disease in a subject.

In some embodiments, a reference level can be a risk reference level, i.e., a level that is correlated with an increased risk of mortality, complications, or length of hospital stay, or can represent risk of progressing to a more severe form of disease, e.g., from SIRS, sepsis or severe sepsis to septic shock. Thus in a subject who presently has SIRS, sepsis or severe sepsis, the presence of levels of Cit H3 above a risk reference level indicates that the subject has an increased risk of mortality, complications, or length of hospital stay, or can represent risk of progressing to a more severe form of disease (e.g., developing septic shock), and should be treated accordingly (e.g., with a treatment known in the art and/or described herein). Complications can include multiple organ dysfunction (e.g., respiratory failure, renal failure, hepatic damage, and/or cardiac dysfunction), secondary infections, and prolonged need for supportive care (e.g., dialysis, ventilator, and/or cardiac drugs), resulting in in longer ICU and hospital stay, more interventions/procedures, increased costs, and worse long-term disability (i.e., poor functional status), thus the level of Cit H3 above a risk reference level can also represent an increased risk of any of these complications or sequelae. Such risk reference levels can be determined by routine methods.

Treating Septic Shock

The methods described herein can include using levels of Cit H3 to select a treatment for subject, e.g., the administration of an effective amount of a pharmaceutical agent for the treatment of septic shock, e.g., an antimicrobial agent. Thus, the methods can administering a treatment for septic shock to a subject as having a level of Cit H3 above a reference level; optionally the methods can include

The terms “effective amount” and “effective to treat,” as used herein, refer to an amount that is effective within the context of its administration for causing an intended effect or physiological outcome. Effective amounts in the present context include, for example, amounts that reduce injury to a specific organ(s) effected by septic shock, or generally improve the patient's prognosis following septic shock. The term “treat(ment)” is used herein to describe delaying the onset of, inhibiting, or alleviating the detrimental effects of a condition, e.g., organ injury/failure associated with or caused by septic shock.

Standard treatments for septic shock include fluid resuscitation and the transfusion of fluids (e.g., with isotonic crystalloids and/or colloids, titrated to a selected central venous pressure (CVP) goal between 8 and 12 mm Hg or signs of volume overload (dyspnea, pulmonary rales, or evidence of pulmonary edema on a chest radiograph), blood and/or blood products; mechanical ventilation; administration of drugs to treat low blood pressure (e.g., administration of inotropics or vasopressors), infection (e.g., antimicrobial therapy), or blood clotting (e.g., Activated Protein C (APC)); oxygen; insulin as needed; and surgical excision or drainage of the infected tissues, if possible). In some embodiments, the treatments include pharmacological blockade of high-mobility group B1 protein (HMGB1), macrophage migration inhibitory factor (MIF), the complement split product, C5a, and apoptosis inhibitors. See, e.g., Dellinger et al., Crit Care Med. January 2008; 36(1):296-327, which is incorporated herein by reference in its entirety, and especially for teachings relating to treatment of septic shock.

For example, the methods can include determining a level of Cit H3 in the subject, and administering a treatment to the subject if the level of Cit H3 is above a preselected reference level or threshold. As one example, the treatment includes administration of an antibiotic or antimicrobial agent. Examples of classes of antibiotics that can be used in the methods described herein include penicillins, cephalosporins, carbacephems, cephamycins, carbapenems, monobactams, quinolones, tetracyclines, aminoglycosides, macrolides, glycopeptides, chloramphenicols, glycylcyclines, licosamides, lipopeptides, oxazolidinones and fluoroquinolones.

Monitoring the Efficacy of Treatments of Septic Shock

The methods described herein can include using levels of Cit H3 to monitor the effectiveness of a treatment for septic shock, e.g., the administration of an effective amount of a pharmaceutical agent for the treatment of septic shock. For example, multiple levels of Cit H3 can be determined over time, and the change in levels is indicative of whether the treatment is effective: a decrease in Cit H3 serum levels over time indicates that the treatment is effective, while no change or an increase indicates that the treatment is not effective. The methods can also be used to monitor changes in risk of progression to a more severe form of disease, e.g., from sepsis or severe sepsis to septic shock; multiple serum levels of Cit H3 can be determined over time, and the change in levels is indicative of whether the treatment is effective in reducing risk: a decrease in Cit H3 serum levels over time indicates that the treatment is effective in reducing risk of progression, while no change or an increase indicates that the treatment is not effective in reducing risk of progression.

Measuring Levels of Cit H3

The methods described herein can include determining a level of Cit H3 in a sample from a subject, e.g., a sample comprising a biological fluid. In some embodiments of the present methods, the sample is or includes serum (e.g., whole blood, serum, or plasma), cerebrospinal fluid, urine, saliva, peritoneal fluid, or a portion or subfraction thereof. In some embodiments, the sample is or includes serum or a portion or subfraction thereof. In some embodiments, the sample is or includes urine or a portion or subfraction thereof. The presence and/or level of Cit H3 can be evaluated using methods known in the art, e.g., using quantitative immunoassay methods such as enzyme linked immunosorbent assays (ELISAs), immunoprecipitations, immunofluorescence, enzyme immunoassay (EIA), radioimmunoassay (RIA), and Western blot analysis. In some embodiments, the methods include contacting an agent that selectively binds to the Cit H3 protein (such as an antibody or antigen-binding portion thereof) with a sample, to evaluate the level of protein in the sample. In some embodiments, the antibody bears a detectable label. Antibodies can be polyclonal, or more preferably, monoclonal. An intact antibody, or an antigen-binding fragment thereof (e.g., Fab or F(ab′)2) can be used. The term “labeled,” with regard to an antibody encompasses direct labeling of the antibody by coupling (i.e., physically linking) a detectable substance to the antibody, as well as indirect labeling of the antibody by reactivity with a detectable substance. Examples of detectable substances are known in the art and include chemiluminescent, fluorescent, radioactive, or colorimetric labels. For example, detectable substances can include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In some embodiments, high throughput methods, e.g., protein or gene chips as are known in the art (see, e.g., Ch. 12, “Genomics,” in Griffiths et al., Eds. Modern genetic Analysis, 1999, W. H. Freeman and Company; Ekins and Chu, Trends in Biotechnology, 1999; 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of Cit H3.

In some embodiments, microfluidic (e.g., “lab-on-a-chip,” “micro-a-fluidic chips”) devices can be used in the present methods for detection and quantification of Cit H3 protein in a sample. Such devices have been successfully used for microfluidic flow cytometry, continuous size-based separation, and chromatographic separation. In particular, such devices can be used for the isolation of specific biological particles such as specific proteins (e.g., Cit H3) from complex mixtures such as serum (e.g., whole blood, serum, or plasma), cerebrospinal fluid, urine, saliva, or peritoneal fluid. A variety of approaches may be used to separate Cit H3 proteins from a heterogeneous sample. For example, some techniques can use functionalized materials to capture Cit H3 using functionalized surfaces that bind to the target cell population. The functionalized materials can include surface-bound capture moieties such as antibodies or other specific binding molecules, such as aptamers, as are known in the art. Accordingly, such microfluidic chip technology may be used in diagnostic and prognostic devices for use in the methods described herein. For examples, see, e.g., Lion et al., Electrophoresis 24 21 3533-3562 (2003); Fortier et al., Anal. Chem., 77(6):1631-1640 (2005); U.S. Patent Publication No. 2009/0082552; and U.S. Pat. No. 7,611,834. Also included in the present application are microfluidics devices comprising Cit H3 binding moieties, e.g., anti-Cit H3 antibodies or antigen-binding fragments thereof.

### EXAMPLES

The invention is further described in the following examples, which do not limit the scope of the invention described in the claims.

The following antibodies and supplies were used in the studies described herein. LPS (from S. typhosa, Cat# L6386, Lot#038k4005) and dimethyl sulfoxide (DMSO) were purchased from the Sigma Aldrich, Co (St. Louis, Mo.). Suberoylanilide hydroxamic acid was purchased from Enzo Life Sciences International, Inc (Plymouth Meeting, Pa.). Primary antibody against acetyl-histone H3 at lysine 9 (Ac H3) was purchased from Upstate (Lake Placid, N.Y.). Anti-citrulinated histone H3 (citrulline 2+8+17) antibody was purchased from abcam (Cambridge, Mass.). Anti-actin antibody was purchased from Sigma-Aldrich, Co. Anti-mouse and anti-rabbit IgG secondary antibodies were purchased from Amersham Biosciences (Piscataway, N.J.). Protease Inhibitor Cocktail II was purchased from Calbiochem (San Diego, Calif.). RPMI 1640 medium, fetal bovine serum (FBS), and phosphate buffered saline (PBS) were from Gibco-BRL (Grand Island, N.Y.). L-glutamine, and fetal calf serum (FCS) were from Invitrogen (Carlsbad, Calif.). All-trans retinoic acid (ATRA) was purchased from Acros Organics (Geel, Belgium). All other chemicals in this study were of analytical grade and obtained from the Sigma-Aldrich if not mentioned otherwise.

In the present studies, statistical differences were determined by Student t tests and ANOVA for two group and multiple group comparisons respectively (SPSS statistical software package, Chicago, Ill.). Kaplan-Meier survival curves were analyzed by using the MedCalc Statistical Software (Mariakerke, Belgium) for the comparison of LPS large dose (LD) and LPS small dose (SD) groups. Fisher's exact test was used to analyze association of serum protein biomarker with lethality of LPS-induced septic shock. Differences were considered to be statistically significant when p values were <0.05.

### Example 1

### Serum Levels of Histone H3 in the Early Stage of Septic Shock in Mice Treated with or without SAHA

To identify biomarkers of septic shock and treatment, isobaric tag labeling for relative and absolute quantitation (iTRAQ) was performed as described in WO 2010/126635 (which is incorporated herein by reference) in sham (no LPS and no treatment), septic shock (3 h after LPS injection) and SAHA treated (5 h after the first SAHA treatment, and 3 h after LPS injection) animals. Several potential serum biomarkers in LPS-induced septic shock and treatment were found. One of them was histone H3, a chromatin protein in nucleus. This protein was elevated in LPS-induced septic shock, and treatment with SAHA decreased histone H3 in serum dramatically.

To assure that the data were reliable, independent serum samples were collected and analyzed. As in a previous study (Li et al., Shock. 2009; 32(5): 517-523), male C57B1/6J mice (n=9, 3/group) weighing 25-30 g (purchased from Jackson Labs (Bar Harbor, Me.)) were randomly divided into two groups: LPS control, and SAHA+LPS. Normal saline (10 μl/g body weight) or LPS (20 mg/kg) dissolved in normal saline was injected intraperitoneally. The mice were given DMSO (1 μl/g body weight) or SAHA (50 mg/kg) dissolved in DMSO intraperitoneally 2 hours (h) before, and then soon after injection of LPS. Sham mice (no LPS, no SAHA) were used as the control. Serum separated from these blood samples was saved at −80° C. and used for the current experiment. In the present study, a large dose (35 mg/kg) or a small dose (10 mg/kg) of LPS was administrated intraperitoneally. All animals were housed in plastic cages and had access to chow and water throughout the experiment. The animals were kept at room temperature (24±2° C.) and exposed to alternative cycles of 12 h light and darkness. Survival rate was then recorded for over the next 7 days. Blood was collected from the orbital sinus (as described in Hoff, Lab Animal. 2000; 29(10): 47-53) at 3 h after LPS injection.

The samples were analyzed by Western blot with anti-histone H3 antibody. Western blot analysis was performed as follows. Proteins (about 100 μg per lane) were separated by sodium dodecyl sulphate-polyacrylamide gel electrophoresis (SDS-PAGE) on 15% polyacrylamide gels and transferred onto nitrocellulose membranes (Bio-Rad Laboratories, Hercules, Calif.). The membranes were blocked in 0.05% PBS-Tween (PBST) containing 5% milk (Bio-Rad Laboratories, Hercules, Calif.) and then incubated with the primary antibody at 4° C. overnight. The primary antibody was detected by incubation with horseradish peroxidase-coupled second antibody (1:3,000 in PBST with 5% milk) at room temperature for 2 h. The chemiluminescence detection was performed by using WesternLighting Chemiluminescence Reagent Plus (PerkinElmer LAS, Inc., Boston, Mass.). Films were developed using a standard photographic procedure and quantitative analysis of detected bands was carried out by densitometer scanning using VersaDoc Imaging System (BioRad Laboratories, Hercules, Calif.).

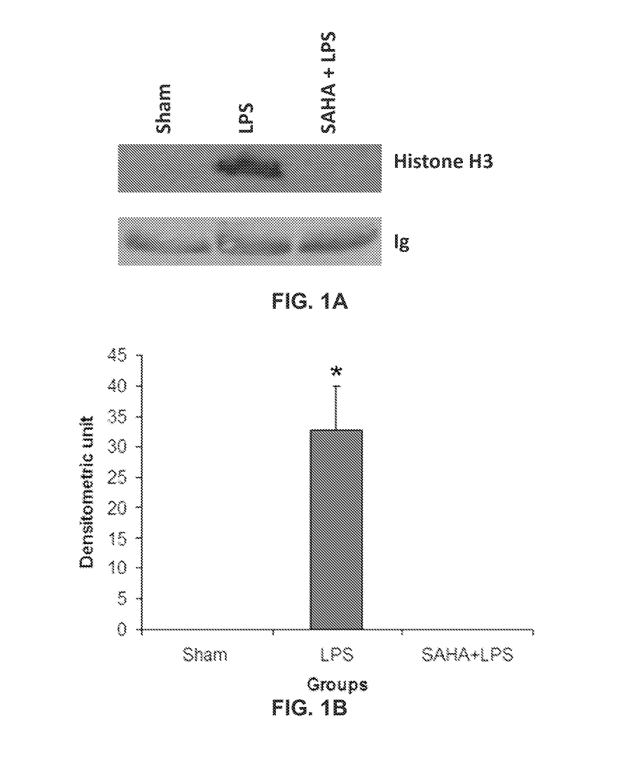

In serum from sham and SAHA treatment groups, no histone H3 was detected. However, a histone H3 band was clearly observed from septic shock group (FIGS. 1A-B). These results demonstrated that septic shock significantly increased serum levels of histone H3, and SAHA treatment prevented this elevation. This raised several questions, such as the source of histone H3 in the circulation and whether serum levels could be used to assess the severity of sepsis.

### Example 2

### Deiminated/Citrullinated Histone H3 and Histone H3 in LPS-Stimulated HL-60 Granulocytes

Previous studies have demonstrated that histone proteins can be deiminated/citrullinated by PAD4 in neutrophils in response to bacterial infection, and the histone hypercitrullination mediates chromatin decondensation and neutrophil extracellular trap formation (Wang et al., J Cell Biol. 2009; 184(2): 205-213). Neutrophils might be a source of serum histone H3 during sepsis. To test whether histone H3 deimination/citrullination is induced as part of the neutrophil response to bacterial endotoxin, HL-60 cells obtained from American Type Culture Collection (ATCC) were maintained in Iscove's modified DMEM medium (Invitrogen Life Technologies, Carlsbad, Calif.) supplemented with 20% fetal bovine serum (FBS). These cells were grown at 37° C. in a humidified incubator in 5% CO2 and 95% air, and differentiated into granulocytes by culturing the cells in medium containing 1 μM ATRA for 3 days. The ATRA-differentiated HL-60 granulocytes were treated with 4 μM calcium ionophore in medium containing 1.5 mM calcium chloride, and then incubated with LPS (100 ng/ml) in the presence or absence of SAHA (10 μM) over 3 h. Following incubation, medium was collected, and cell lysates were prepared. The medium and the lysates were tested for H3, Ac H3, Cit H3 and actin (internal control) by Western blots. To ensure there was no false positive data that may be produced from the medium with dead cells, the cells were carefully treated and a sham group was also set up as a control.

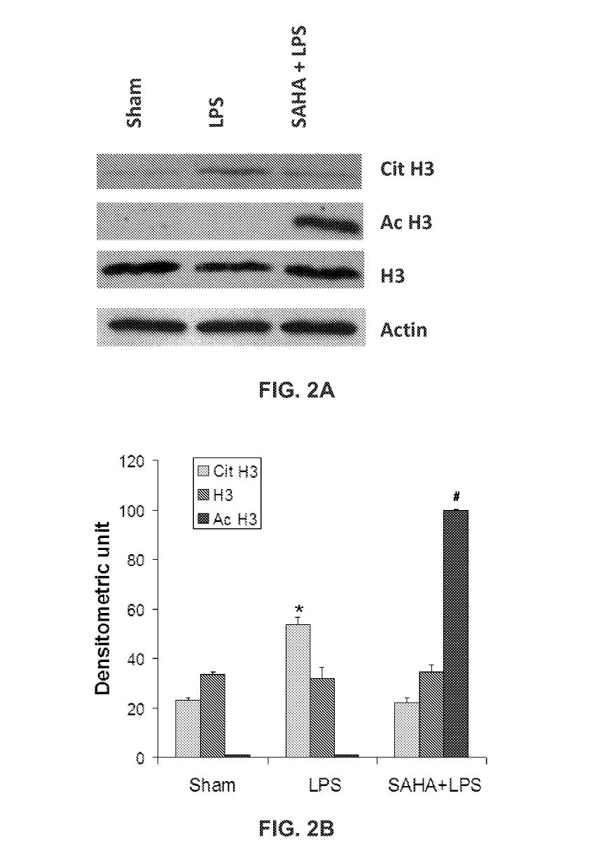

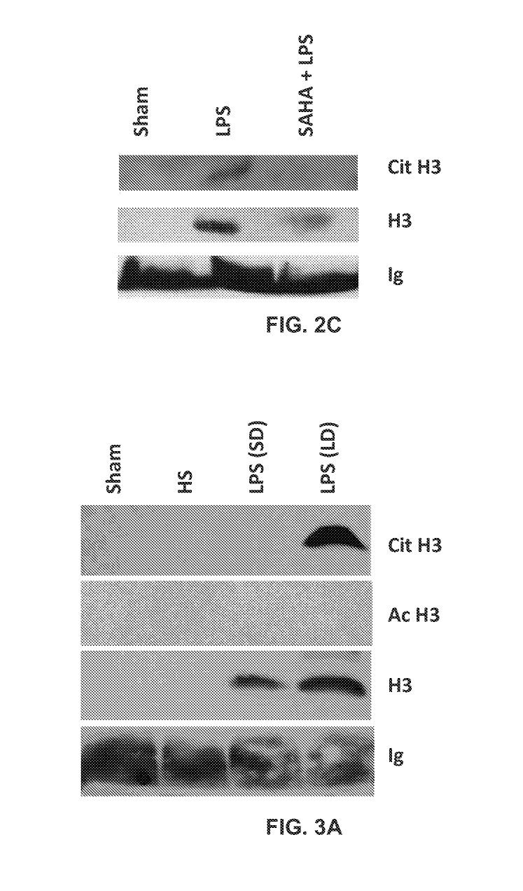

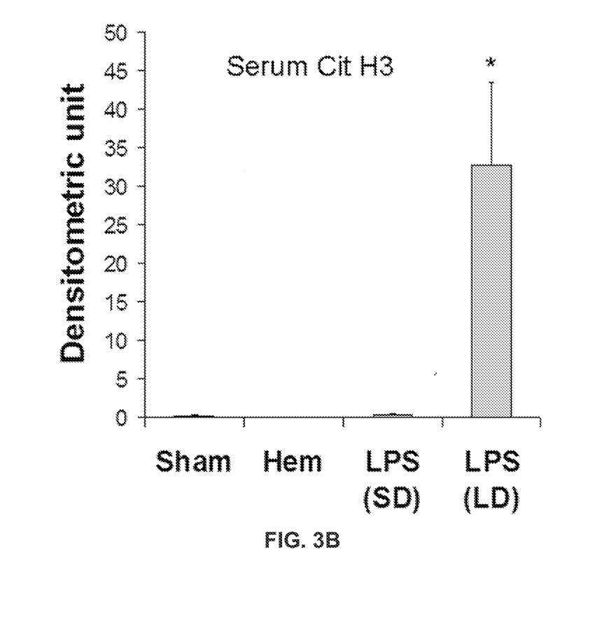

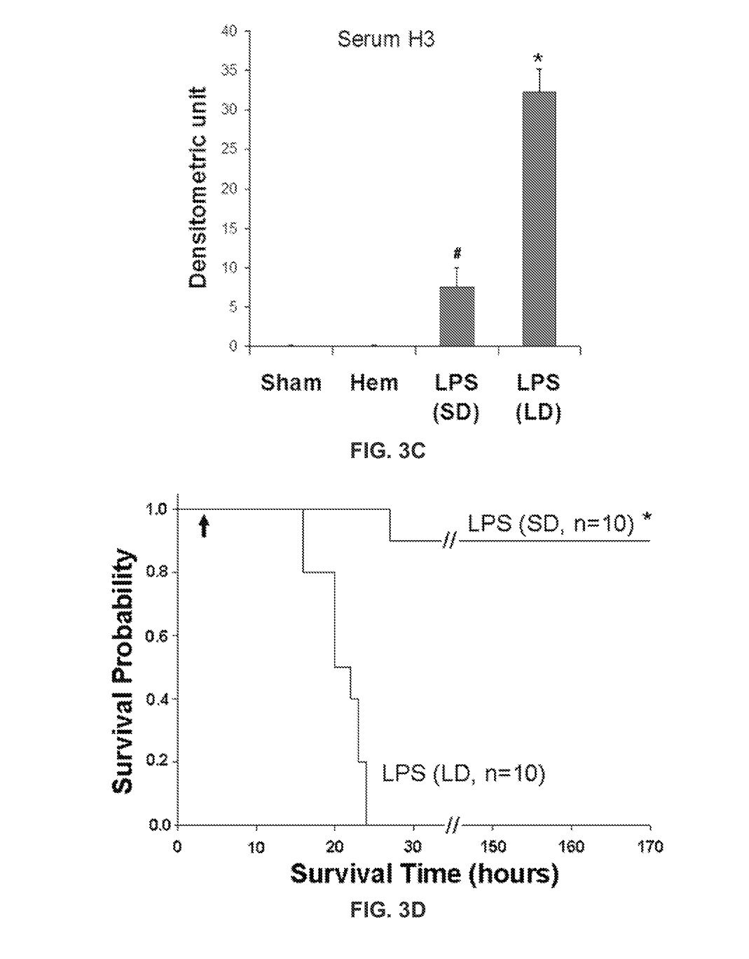

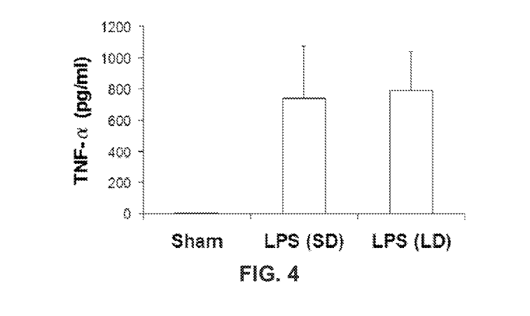

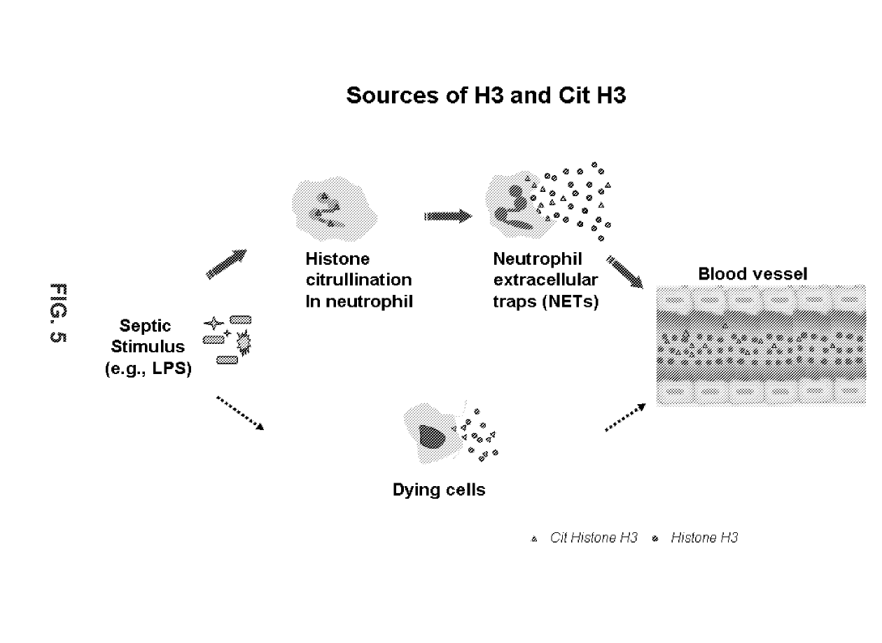

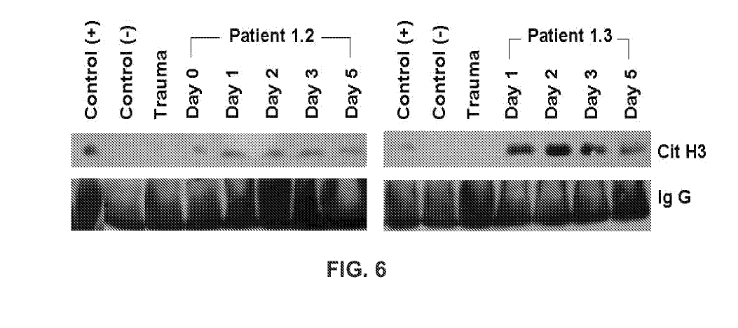

As shown in FIGS. 2A-B, an increase in Cit H3 was detected in the LPS group compared to the sham control. SAHA treatment significantly reduced the expression of Cit H3 along with an increase in acetylated H3. Moreover, more Cit H3 and H3 were observed in the cultured cell medium of LPS group (FIG. 2C). By contrast, there were no detectable Cit H3 and H3 proteins in the medium from the SAHA+LPS group. These results suggest that activation of granulocytes by LPS is likely one of the sources of H3 and Cit H3, and LPS-induced H3 citrullination could play an important role in the release of histone H3 into extracellular space. SAHA decreased H3 citrullination and reversed these alterations.

### Example 3

### Effect of Different LPS Doses on Serum Levels of Cit H3 and H3

It has been reported that the effects of LPS on lethality and protein expression are dose-dependent (Barton and Jackson, Infect Immun. 1993; 61(4): 1496-1499; Haque et al., Pneumon. 2009; 22(2): 143-155). To determine whether serum levels of H3, Cit H3 and acetylated H3 were dependent on the LPS dose, the mice were injected with a small dose (10 mg/ml) or a large dose (35 mg/ml). The serum levels of all three histone H3 were then measured and correlated to mortality. Mice were randomly divided into three groups (n=10/group): (1) sham group (no LPS), (2) LPS small dose (SD) group, and (3) LPS large dose (LD) group. Blood was collected at 3 h after LPS injection and serum was prepared for immunoblotting. All animals were observed for the survival rate.

As shown in FIG. 3D, in the LPS-induced septic shock condition, all mice from LPS large dose (LD) group died in less than 23 h. However, almost all of the LPS small dose (SD) animals displayed long term survival. Only one of ten mice died at 27 h after LPS injection, while the rest survived for the entire 7 day observation period. The survival rate in the LPS (SD) group was significantly better compared to the LPS (LD) animals (90% vs. 0% respectively). To determine whether Cit H3 might be used as a biomarker to assess severity of LPS-induced septic shock, serum samples from each group were analyzed (n=5/group). Serum samples from mice that underwent hemorrhagic shock (HS) served as a control for the biomarker specificity. The Western blot data showed that serum levels of Cit H3 and H3 were high in the LD group mice, whereas these proteins were not detectable in the serum obtained from the sham and HS groups (FIGS. 3A and 3B). An increase in serum H3 was found in LPS (SD) animals but not in sham (p<0.038) and HS groups (p<0.016), although the induction was lower than that from LPS (LD) group (p<0.009) (FIGS. 3A and 3C). No Ac H3 was detected in all animals (FIG. 3A). These results indicate that lethal and sub-lethal doses of LPS induce very different serum profiles, with a dose dependent increase in the circulating Cit H3 and H3 levels. Also, these data imply that Cit H3 is a specific biomarker for severe LPS-induced septic shock among these groups.

### Example 4

### Serum Levels of Cit H3 are Associated with Severity of LPS-Induced Septic Shock

The average survival times were about 20 h and 146 h for the LD and SD LPS groups respectively. The earliest time of death was ˜16 h and 27 h, whereas the longest survival times were 23 h and 168 h for LD and SD groups respectively. The only mouse that died in the SD group still survived longer (27 h) than the best survivors (23 h) in the LD group. Most importantly, serum Cit H3 was high in the LD group and undetectable in the SD group (Table 1). Statistical analysis with Fisher's exact test showed that the survival difference was significant between LPS (SD) and LPS (LD) groups (p=0.00006, n=10). These results indicate that an early increase in circulating Cit H3 protein is associated with high lethality in this model of LPS-induced shock.

TABLE 1
Serum Cit H3 and H3 associate with severity of LPS-induced sepsis

| | LPS | | Fisher's |
|---|---|---|---|
| | (LD, n = 10) | LPS (SD, n = 10) | Exact Test |
| Serum H3 | + | +/− | |
| Serum Cit H3 | + | − | * p = 0.00006 |
| Mice died within 23 h | 10 | None | |
| Mice died at 27 h | N/A | 1 | |
| Mice survived over | N/A | 9 | |
| 168 h | | | |

### Example 5

### Serum Levels of TNF-α Protein in Mice Insulted by Different Dose of LPS In Vivo

There is general agreement that the main pathogenic mediator in lethal septic shock is TNF-α (Beutler and Cerami, Annu Rev Immunol. 7: 625-55, 1989). To determine whether TNF-α can predict lethality of LPS-induced shock as well as Cit H3, blood was collected from sham, LPS (SD), and LPS (LD) groups at 3 h after LPS injection (n=5/group). Quantitative determination of TNF-α in the blood of mice was made using Quantikine Enzyme-Linked Immunosorbent Assay (ELISA) Kit (R&D Systems, Minneapolis, Minn.) according to manufacturer's instruction. The concentration of the cytokine was measured by optical densitometry at 450 nm in a SpectramaxPlus 384 microplate reader (Molecular Devices, Sunnyvale, Calif.). All of the analyses were performed in triplicates.

Serum TNF-α protein was hardly detected in the normal mice, but LPS injection increased the circulating level of TNF-α protein. At 3 h after endotoxin insult, TNF-α increased to 741 pg/ml in LPS (SD) group and 792 pg/ml in LPS (LD) group. Statistical analysis showed that there was no significant difference in serum TNF-α levels between these two groups (FIG. 4; p=0.77). These results demonstrate that TNF-α release in response to LPS occurs in a non-dose-dependent fashion. Together with the data described above, these studies indicate that serum Cit H3 does not simply mimic the levels of TNF-α, but may better reflect the severity of shock.

### Example 6

An ongoing clinical study has shown an increase in circulating Cit H3 levels in patients with ventilator associated pneumonia due to multiple organisms.

Healthy Subjects:

Inclusion criteria: Age ≧18; able to give consent; usual state of good health; ≧110 lbs.

Exclusion criteria: Age <18; chronic inflammatory disease; acute illness; immunosuppressive drugs; AIDS; malignancies; transfusion or blood products within the past 24 hours; <110 lbs; blood donation in the last 3 weeks.

Trauma Patients:

Inclusion criteria: Major trauma patient. Injury Severity Score (ISS)>16; Age ≧18; ≧110 lbs;

Exclusion criteria: Age <18; chronic inflammatory disease; immunosuppressive drugs; AIDS; malignancies; <110 lbs; known significant phlebotomy, e.g. >100 ml, in the past 3 weeks for care of blood bank.

Septic Patients:

Inclusion criteria: Major trauma patient. Injury Severity Score (ISS)>16; Age ≧18; ≧110 lbs; and during hospital stay, clinical diagnosis of sepsis according to the criteria of the American College of Chest Physicians/Society of Critical Care Medicine.

Exclusion criteria: Age <18; chronic inflammatory disease; immunosuppressive drugs; AIDS; malignancies; transfusion or blood products within the past 24 hours; <110 lbs; known significant phlebotomy, e.g. >100 ml, in the past 3 weeks for care of blood bank.

To date 21 samples have been collected from healthy volunteers, 4 samples from 1 trauma patient, and 9 samples from 2 septic patients. Western blots were performed on these samples with a monoclonal antibody against Cit H3 as follows. Equal amounts of plasma were separated by sodium dodecyl sulphate-polyacrylamide gel electrophoresis (SDS-PAGE) on 15% polyacrylamide gels and transferred onto nitrocellulose membranes (Bio-Rad Laboratories, Hercules, Calif.). The membranes were blocked in 0.05% PBS-Tween (PBST) containing 5% milk (Bio-Rad Laboratories, Hercules, Calif.) and then incubated with the antibody against Cit H3 (citrulline 2+8+17), purchased from Abcam (Cambridge, Mass.), at 4° C. overnight. The primary antibody was detected by incubation with horseradish peroxidase-coupled second antibody (1:3,000 in PBST with 5% milk) at room temperature for 2 h. The chemiluminescence detection was performed by using WesternLighting Chemiluminescence Reagent Plus (PerkinElmer LAS, Inc., Boston, Mass.). Films were developed using a standard photographic procedure and quantitative analysis of detected bands was carried out by densitometer scanning using VersaDoc Imaging System (BioRad Laboratories, Hercules, Calif.).

Cit H3 is not expected to be present at significant or detectable levels in the circulation of normal, healthy humans. Therefore, blood from a mouse endotoxic shock model and from human healthy subjects were used as positive and negative controls, respectively.

As shown in FIG. 6, Cit H3 was not detected in normal human blood. Interestingly, no Cit H3 protein was detected in a blood sample from a patient with trauma hemorrhage either. However, sepsis dramatically induced blood levels of Cit H3 compared to healthy volunteers and hemorrhagic patients. These results indicate that Cit H3 levels are increased in septic patients. Furthermore, it is likely that this response is specific to septic shock, as it was not seen in the hemorrhagic trauma patient.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An in vitro method of predicting prognosis of a subject suffering from septic shock (SS), the method comprising:
determining a level of Cit H3 in a sample comprising serum from the subject to obtain a test value; and
comparing the test value to a reference value,
wherein a test value compared to the reference value indicates the subject's prognosis.

2. The method of claim 1, wherein the sample comprises plasma or whole blood.

3. The method of claim 1, wherein the reference value represents a threshold level of Cit H3, wherein the presence of a level of Cit H3 in the subject that is above the reference value indicates that the subject has an increased risk of mortality, complications, or longer hospitalization due to septic shock, and the presence of a level of Cit H3 in the subject that is below the reference value indicates that the subject has an increased chance of survival.

4. The method of claim 1, wherein the subject is a human.

5. An in vitro method of predicting prognosis of a subject suffering from Systemic Inflammatory Response Syndrome (SIRS), sepsis, or severe sepsis, the method comprising:
determining a level of Cit H3 in a sample comprising serum from the subject to obtain a test value; and
comparing the test value to a reference value,
wherein a test value compared to the reference value indicates the subject's prognosis.

6. The method of claim 5, wherein the sample comprises plasma or whole blood.

7. The method of claim 5, wherein the reference value represents a threshold level of Cit H3, wherein the presence of a level of Cit H3 in the subject that is above the reference value indicates that the subject has an increased risk of progressing to severe sepsis or septic shock, and the presence of a level of Cit H3 in the subject that is below the reference value indicates that the subject does not have an increased risk of progressing to severe sepsis or septic shock.

8. The method of claim 5, wherein the subject is a human.

9. An in vitro method of diagnosing sepsis, severe sepsis, or septic shock in a subject suffering from Systemic Inflammatory Response Syndrome (SIRS), the method comprising:
determining a level of Cit H3 in a sample comprising serum from the subject to obtain a test value; and
comparing the test value to a reference value,
wherein a test value compared to the reference value indicates whether the subject has sepsis, severe sepsis, or septic shock.

10. The method of claim 9, wherein the sample comprises plasma or whole blood.

11. The method of claim 9, wherein the reference value represents a threshold level of Cit H3, wherein the presence of a level of Cit H3 in the subject that is above the reference value indicates that the subject has septic shock, and the presence of a level of Cit H3 in the subject that is below the reference value indicates that the subject does not have septic shock.

12. The method of claim 9, wherein the subject is a human.

13. The method of claim 11, further comprising selecting a subject who has a level of Cit H3 in the subject that is above the reference value, and administering one or more treatments for septic shock to the subject.

14. The method of claim 13, wherein the treatment comprises administering a therapeutically effective amount of an antibiotic.

15. An in vitro method of evaluating the efficacy of a treatment for septic shock (SS) in a subject, the method comprising:
determining a level of Cit H3 in a first sample comprising serum from the subject to obtain a first value;
administering a treatment for septic shock to the subject;
determining a level of Cit H3 in a subsequent sample comprising serum obtained from the subject at a later time, to obtain a treatment value; and
comparing the first value to the treatment value,
wherein a treatment value that is below the first value indicates that the treatment is effective.

16. The method of claim 15, wherein the first and second samples comprise plasma or whole blood.

17. The method of claim 15, wherein the treatment includes administration of an effective amount of an antibiotic.

18. The method of claim 15, wherein the subject is a human.

19-26. (canceled)
